(19) United States
(12) Patent Application Publication
(10) Pub. No.: US 2018/0296820 A1
(43) Pub. Date: Oct. 18, 2018

(54) STOPCOCK

(71) Applicant: Smiths Medical ASD, Inc., Plymouth, MN (US)

(72) Inventors: Kevin Bogoslofski, Southington, CT (US); Jay T. Breindel, Southington, CT (US); Thomas T. Koehler, Simsbury, CT (US); Christopher Roehl, New Hartford, CT (US)

(21) Appl. No.: 15/951,899

(22) Filed: Apr. 12, 2018

Related U.S. Application Data
(60) Provisional application No. 62/485,016, filed on Apr. 13, 2017.

(51) Int. Cl. A61M 39/22; F16K 5/04; F16K 11/083; F16K 11/085; F16K 27/06; A61M 39/26

(52) U.S. Cl. CPC A61M 39/223; F16K 5/0407; F16K 11/0833; F16K 11/0853; A61J 1/2062; A61M 39/26; A61M 2039/229; A61M 2039/263; F16K 27/06

(57) ABSTRACT

A stopcock and sampling port device. The stopcock and sampling port device configured to reduce the occurrence of stagnation fluid flowing therethrough, the stopcock and sampling port device including a housing, an elastomeric element, a cap, a handle, and a divided septum, the housing defining an internal fluid passageway having a first port, a second port, and a third port, the divided septum operably coupled to the housing within the third port and configured to encourage turbulence of fluid passing through the third port, thereby reducing the occurrence of stagnation of fluid flowing therethrough.

### RELATED APPLICATION

The present application claims the benefit of U.S. Provisional Application No. 62/485,016 filed Apr. 13, 2017, which is hereby incorporated herein in its entirety by reference.

### TECHNICAL FIELD

The present disclosure relates generally to stopcocks. More particularly, the present disclosure relates to stopcocks in which the stagnation of fluid flowing therethrough is minimized.

### BACKGROUND

In the clinical setting, there is frequently a need to monitor and evaluate a patient's blood chemistry. One of the most common methods of obtaining a blood sample from a patient is through the use of a sharpened cannula. According to this method, a sharpened cannula is inserted into a vein or artery of the patient. Thereafter, a blood sample is obtained, for example, the patient's blood can enter into a collection vile under its own pressure, or the blood can be extracted by a syringe. Once the appropriate volume of blood has been obtained, the sharpened cannula is removed from the patient and disposed of.

During the treatment of certain conditions, some patients may require blood sampling as many as twelve times per day. Such frequent sampling potentially exposes the patient to airborne bacteria and viruses which can enter the bloodstream through the opening made by the sharpened cannula. Moreover, each handling of a sharpened cannula presents the risk of an inadvertent needlestick by a clinician. The problem of infection, accidental needle sticks and the ubiquitous danger of contracting blood-borne diseases, such as HIV and/or hepatitis, prompted the medical field to seek alternative blood sampling methods.

One such method is to obtain multiple blood samples from a catheter inserted into the patient. Examples of catheters include central venous line catheters which, for example, can be placed into the right subclavian vein, or arterial line catheters which can be inserted into an artery. Typically, the injection sites for arterial and central venous line catheters are used for drug infusion and pressure monitoring, in addition to blood sampling.

In order to avoid sampling blood mixed with infusion fluid, early blood sampling via arterial and central venous line catheters required a two-step process. In the first step, a first volume of fluid (generally between about 3-10 mL) was withdrawn into a first syringe. As the first volume of fluid was known to include some infusion fluid, it was generally considered unreliable for blood chemistry measurements, and therefore was discarded. After the first volume of fluid had been cleared, in the second step, a second syringe was connected to the catheter and a second volume of fluid was withdrawn from the artery or vein for evaluation.

In addition to the unnecessary loss of blood, the connection and disconnection of the first and second syringes of the two-step process had the potential of introducing air and contaminants into the blood supply. Accordingly, blood sampling systems in which the first volume of fluid could be temporarily stored and reinjected into the patient after the second step was developed.

Referring to FIG. 1A, a conventional blood sampling system 32 is depicted. Conventional blood sampling system 32 includes a distal end 34, which can include a Luer connector (not depicted) for connection to a catheter or other conduit leading to a patient. A first tubing line 36 extends between the distal end 34 and a first stopcock 38. The first stopcock 38 can include a sampling port assembly 39 configured to enable a clinician to withdraw a fluid sample from the patient. A second tubing line 40 extends between the first stopcock 38 and a second stopcock 42. The second stopcock 42 is attached to the bottom end of a reservoir 44, the reservoir 44 including a reservoir body 46 and a plunger assembly 48. For additional support, the reservoir 44 can be mounted to a bracket 50, which in turn can be secured to a conventional pole supported in a vertical orientation. A third tubing line 52 extends from the second stopcock 42 and terminates at a proximal end 54, which can include a Luer connector (not depicted) for connection to at least one of a fluid supply and/or a blood pressure monitoring system. For example, the blood pressure monitoring system may include a pressure transducer in direct fluid communication with the arterial or venous systems of the patient through tubing lines 36, 40 and 52.

Referring to FIG. 2, an angled, exploded view of the first stopcock 38 and sampling port assembly 39 is depicted. Additional views of the first stopcock 38 and sampling port assembly 39 are depicted in U.S. Pat. No. 7,984,730, the contents of which are incorporated by reference herein. The first stopcock 40 includes a housing 64, a main tubular portion 66 defining a first side port 68, a second side port 70, and a third side port 72. A handle element 74 is arranged so that a portion 80 of the handle element 74 is seated within the main tubular portion 66 of the housing 64. The portion 80 defines a pair of channels or grooves 82a/b, with a divider 84 position therebetween. In some stopcocks 38, the divider 84 is scalloped to enable the passage of fluid between groove 82a and groove 82b when the divider 84 is positioned against a contiguous surface (e.g., as depicted in FIG. 3A). In some stopcocks 38, the scallop of the divider is sufficiently large to enable the monitoring of blood pressure (e.g., via a pressure transducer) when the divider 84 is positioned against a contiguous surface. The sampling port assembly 39 can be comprised of an elastomeric element 76 held in position relative to third side port 72 of housing 64 by a cap 78. Some stopcocks 38 further include a septum dividing the third side port 72 (not depicted).

Referring to FIGS. 3A-D, cross-sectional views of the first stopcock 38 and sampling port assembly 39 are depicted. FIG. 3A depicts the first stopcock 38 in a first position, in which fluid is permitted to flow from the patient through the first side port 68, through groove 82b, past scalloped divider 84, through groove 82a, and through second side port 70. In the first position, fluid flow to the sampling port assembly 39 is inhibited. FIG. 3B depicts the first stopcock 38 in a second position, in which passage of fluid to and from the first side port 68 is inhibited. In this position, fluid from the second side port 70 can be withdrawn through the sampling port assembly 39. FIG. 3C depicts the first stopcock in a third position, in which fluid is permitted to flow from the patient through the first side port 68, through groove 82b, into the third side port 72 (thereby moving fluid through an internal portion of the sampling port assembly 39), through groove 82a, and through the second side port 70. FIG. 3D depicts the first stopcock in a fourth position, in which passage of fluid to and from the second side port 70 is inhibited. In this position, fluid from the patient via first side port 68 can be withdrawn through the sampling port assembly 39. The second stopcock 42 can be configured to operate in a similar manner.

Referring to FIG. 4A, in operation, the conventional blood sampling system 32 can be initially presented with both the first stopcock 38 and the second stopcock 42 positioned in the first position, such that fluid is able to flow from the distal end 34 to the proximal end 54, while inhibiting the flow of fluid into the sampling port assembly 39 and the reservoir 44. Thereafter, referring to FIG. 4B, the first stopcock 38 can be rotated to the third position and the second stopcock 42 can be rotated to the second position. Fluid from the patient can then be withdrawn into reservoir 44. As the fluid from the patient flows through tubing 36, 40, the first stopcock 38 directs the fluid through the third side port 72 and the internal portion of the sampling port assembly 39. Referring to FIG. 4C, the first stopcock 38 can then be rotated to the fourth position, thereby inhibiting the flow of fluid to or from the reservoir 44. A sample of blood can then be withdrawn from the sampling port assembly 39, for example via a syringe 86. Referring to FIG. 4D, the syringe 86 can be removed and the first stopcock 38 can be returned to the second position. Thereafter, the fluid previously withdrawn into reservoir 44 can be reintroduced into the patient via the second stopcock 42. As the fluid from reservoir 44 is reintroduced, the first stopcock 38 will direct the fluid through the third side port 72 and the internal portion of the sampling port assembly 39. Referring to FIG. 4E, both the first stopcock 38 and the second stopcock 42 can be rotated to the third position, such that fluid is again able to flow from the distal and 34 to the proximal end 54, while directing the flow of fluid through the internal portion of the sampling port assembly 39 and the reservoir 44.

Through use of such conventional blood sampling systems 32, it has been found that blood and other fluid has a tendency to stagnate within portions of conventional blood sampling systems 32. The stagnated blood can, among other things, present a breeding ground for bacteria. Within conventional blood sampling systems 32, the most likely place for blood stagnation to occur is within the sampling port assembly 39.

### SUMMARY OF THE DISCLOSURE

Embodiments of the present disclosure present stopcocks and sampling ports designed to reduce or eliminate the occurrence of blood stagnation through the widening of flow channels, the elimination of dead spaces and corners, and the use of angled and/or split partitions to create a more even flow of blood within the stopcocks and sampling ports.

One embodiment of the present disclosure provides a stopcock and sampling port device configured to reduce the occurrence of stagnation of fluid flowing therethrough. The stopcock and sampling port device can include a housing, elastomeric element, cap, handle, and septum. The housing can define a housing internal fluid passageway having a first port, a second port, and third port. The elastomeric element can define an elastomeric element internal fluid passageway and an aperture. The elastomeric element can be configured to bias the aperture closed in a relaxed position, and bias the aperture open in a compressed position. The cap can be configured to operably couple the elastomeric element to the housing. The handle can be rotatably position within the housing internal fluid passageway and can be configured to selectively enable flow between the first port and the second port via the elastomeric element internal fluid passageway. The septum can be operably coupled to the housing within the third port and can extend into the elastomeric element internal fluid passageway. The septum can be configured to encourage fluid passing through the third port to flow through the elastomeric element internal fluid passageway, thereby reducing the occurrence of stagnation of fluid within the elastomeric element internal fluid passageway.

In one embodiment, the septum is divided so as to encourage turbulence in fluid passing through the third port. In one embodiment, at least one of the septum and/or a divider defined by the handle can be shaped and/or angled so as to impart a swirling motion of fluid passing through the third port. In one embodiment at least a portion of the third port is partially occluded so as to impart turbulence a fluid passing therethrough. In one embodiment, the elastomeric element internal fluid passageway and the third port of the housing are shaped and sized to create a smooth transition therebetween. In one embodiment, the elastomeric element internal fluid passageway has a diameter larger than a diameter of at least one of the first port and the second port.

Another embodiment of the present disclosure provides a stopcock and sampling port device configured to reduce the occurrence of stagnation of fluid flowing therethrough. The stopcock and sampling port device can include a housing, elastomeric element, cap, handle, and divided septum. The housing can define a housing internal fluid passageway having a first port, a second port, and a third port. The elastomeric element can define an elastomeric element internal fluid passageway and an aperture. The elastomeric element can be configured to bias the aperture closed in a relaxed position, and bias the aperture open in a compressed position. The cap can be configured to operably couple the elastomeric element to the housing. The handle can be rotatably positioned within the housing internal fluid passageway and can be configured to selectively enable flow between the first port and the second port via the elastomeric element internal fluid passageway. The divided septum can be operably coupled to the housing within the third port. The divided septum can be configured to encourage turbulence of fluid passing through the third port, thereby reducing the occurrence of stagnation of fluid within the elastomeric element internal fluid passageway.

In one embodiment, the divided septum can extend into the elastomeric element internal fluid passageway. In one embodiment at least one of the septum and/or a divider defined by the handle can be shaped and/or angled so as to impart a swirling motion of fluid passing through the third port. In one embodiment, at least a portion of the third port is partially included so as to impart turbulence of fluid passing therethrough. In one embodiment, the elastomeric element internal fluid passageway and the third port of the housing are shaped and sized to create a smooth transition therebetween. In one embodiment, the elastomeric element internal fluid passageway has a diameter larger than a diameter of at least one of the first port and the second port.

Another embodiment of the present disclosure provides a stopcock and sampling port device configured to reduce the occurrence of stagnation of fluid flowing therethrough. The stopcock and sampling port device can include a housing, elastomeric element, cap, handle, and pair of fluid guide vanes. The housing can define a housing internal passageway having a first port, a second port, and a third port. The elastomeric element can define an elastomeric element internal fluid passageway and an aperture. The elastomeric element can be configured to bias the aperture closed in a relaxed position, and bias the aperture open in a compressed position. The cap can be configured to operably couple the elastomeric element to the housing. The handle can be rotatably position within the housing internal fluid passageway, and can be configured to selectively enable flow between the first port and the second port via the elastomeric element internal fluid passageway. The pair of fluid guide vanes can be operably coupled to the housing within the third port. The pair of fluid guide vanes can be configured to impart a swirling motion of fluid passing through the third port, thereby reducing the occurrence of stagnation of fluid within the elastomeric element internal fluid passageway.

In one embodiment, the pair of fluid guide vanes extend into the elastomeric element internal fluid passageway. In one embodiment, the elastomeric element internal fluid passageway and the third port of the housing are shaped and sized to create a smooth transition therebetween. In one embodiment, the elastomeric element internal fluid passageway has a diameter larger than a diameter of at least one of the first port and the second port. In one embodiment, the elastomeric element internal fluid passageway includes a tapered wall, such that a diameter of the elastomeric element internal fluid passageway decreases in proximity to the aperture.

Another embodiment of the present disclosure provides a stopcock and sampling port device configured to reduce the occurrence of stagnation of fluid flowing therethrough. The stopcock and sampling port device can include a housing, elastomeric element, cap, and handle. The housing can include a housing wall defining a housing internal fluid passageway having a first port, second port, and third port. The elastomeric element can include an elastomeric element wall defining an elastomeric element internal fluid passageway and an aperture. The elastomeric element can be configured to bias the aperture closed in a relaxed position, and bias the aperture open in a compressed position. The cap can be configured to couple the elastomeric element to the housing. The handle can be rotatably position within the housing internal fluid passageway and can be configured to selectively enable flow between the first port and the second port via the elastomeric element internal fluid passageway. The elastomeric element wall defining the elastomeric element internal fluid passageway and the housing wall defining the third port can be shaped and sized to create a smooth transition between the elastomeric element internal fluid passageway and the third port, thereby reducing the occurrence of stagnation of fluid within the elastomeric element internal fluid passageway.

In one embodiment, the elastomeric element internal fluid passageway can have a diameter larger than a diameter of at least one of the first port and the second port. In one embodiment, the elastomeric element wall can be tapered, such that a diameter of the elastomeric element internal fluid passageway decreases in proximity to the aperture.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1A is a schematic diagram depicting a blood sampling system of the prior art.

FIG. 2 is an exploded, perspective view depicting a stopcock and sampling port assembly of the prior art.

FIG. 3A is a cross-sectional view depicting the stopcock and sampling port assembly of FIG. 2 in a first position.

FIG. 3B is a cross sectional view depicting the stopcock and sampling port assembly of FIG. 2 in a second position.

FIG. 3C is a cross sectional view depicting the stopcock and sampling port assembly of FIG. 2 in a third position.

FIG. 3D is a cross sectional view depicting the stopcock and sampling port assembly of FIG. 2 in a fourth position.

FIG. 4A-E are a cross-sectional views of the blood sampling system of FIG. 1 in operation.

FIG. 5A is a partial, cross-sectional view depicting a stopcock and sampling port device in accordance with an embodiment of the disclosure, wherein the stopcock and sampling port device has an elastomeric element in a relaxed position.

FIG. 5B is a partial, cross-sectional view depicting the stopcock and sampling port device of FIG. 5B, wherein the elastomeric element is in a compressed position.

FIG. 6 is a partial, cross-sectional view depicting a stopcock and sampling port device, in which a gap is defined between a septum and a handle element, in accordance with an embodiment of the disclosure.

FIG. 7A is a partial, perspective view depicting a stopcock and sampling port device with a first embodiment of a divided septum in accordance with the disclosure.

FIG. 7B is a partial, perspective view depicting a stopcock and sampling port device with a second embodiment of a divided septum in accordance with the disclosure.

FIG. 7C is a partial, perspective view depicting a stopcock and sampling port device with a septum rotated 90° relative to a divider defined within a handle element in accordance with an embodiment of the disclosure.

FIG. 7D is a partial, perspective view of a stopcock and sampling port device with a partially occluded first portion of a third port in accordance with an embodiment of the disclosure.

FIG. 7E is a partial, perspective view of a stopcock and sampling port device with both a partially occluded first portion and partially occluded second portion of a third port in accordance with an embodiment of the disclosure.

FIG. 8 is a partial, cross-sectional view depicting a stopcock and sampling port device with a pair of fluid guide vanes in accordance with an embodiment of the disclosure.

FIG. 9 is a partial, perspective view depicting a stopcock and sampling port device with a pair of fluid guide vein dividers in accordance with an embodiment of the disclosure.

FIG. 10A is a partial, perspective view depicting a first embodiment of a stopcock and sampling port device having a smooth transition between a wall defining an elastomeric element internal fluid passageway and a wall defining a third port in accordance with an embodiment of the disclosure.

FIG. 10B is a partial, perspective view depicting a second embodiment of a stopcock and sampling port device having a smooth transition between a wall defining an elastomeric element internal fluid passageway and a wall defining a third port in accordance with an embodiment of the disclosure.

FIG. 10C is a partial, perspective view depicting a third embodiment of a stopcock and sampling port device having a smooth transition between a wall defining an elastomeric element internal fluid passageway and a wall defining a third port in accordance with an embodiment of the disclosure.

While embodiments of the disclosure are amenable to various modifications and alternative forms, specifics thereof are shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure as defined by the appended claims.

### DETAILED DESCRIPTION

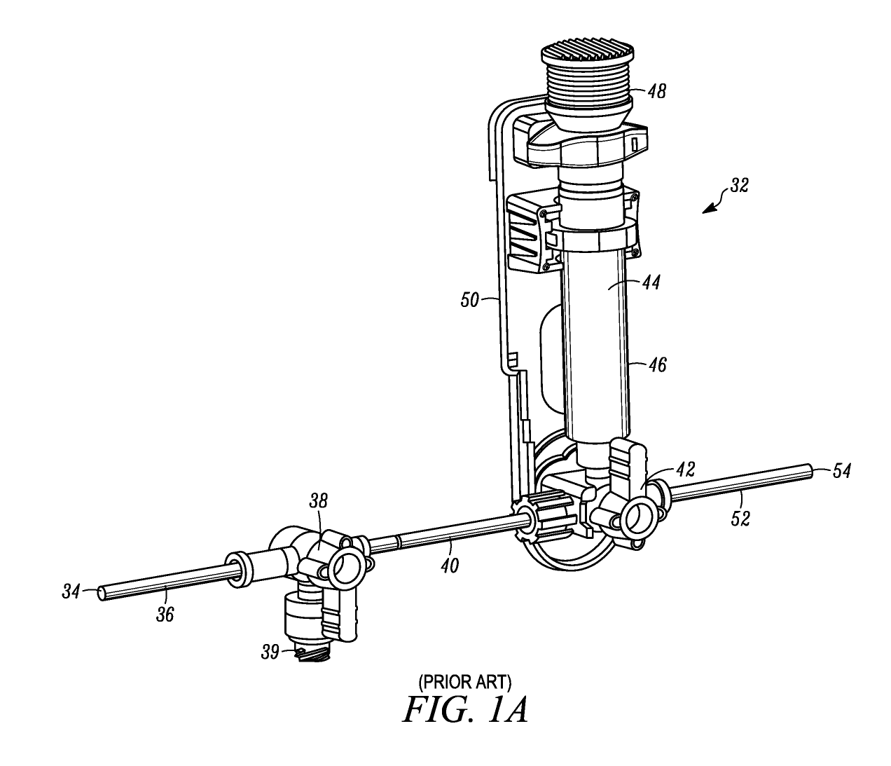

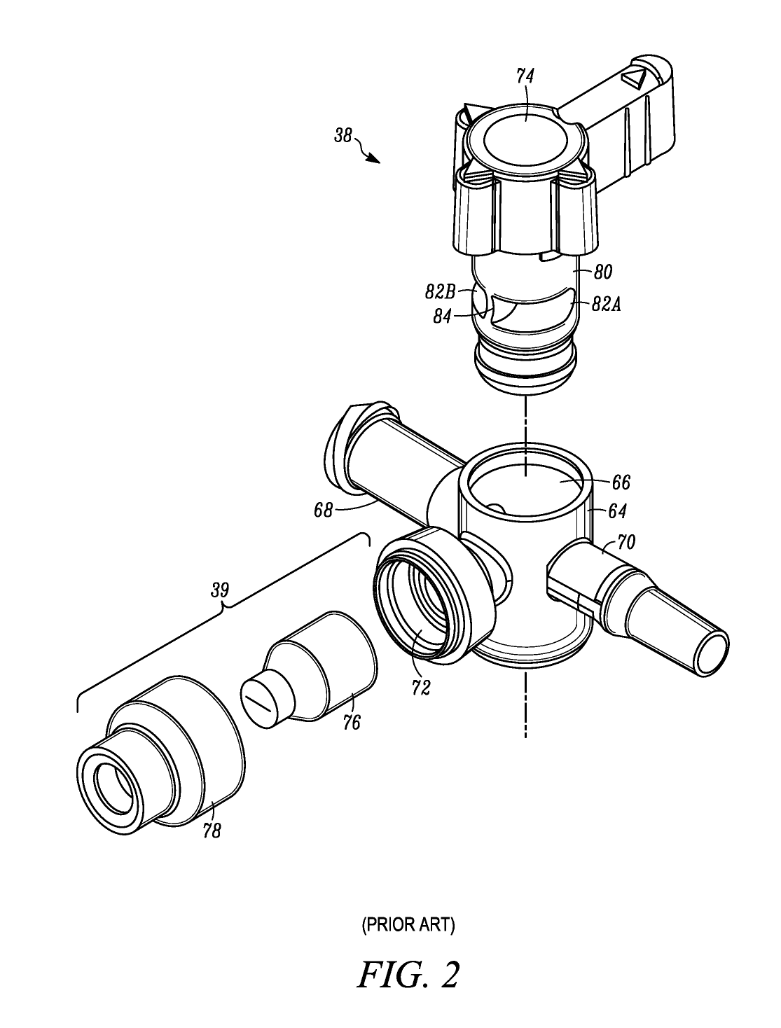

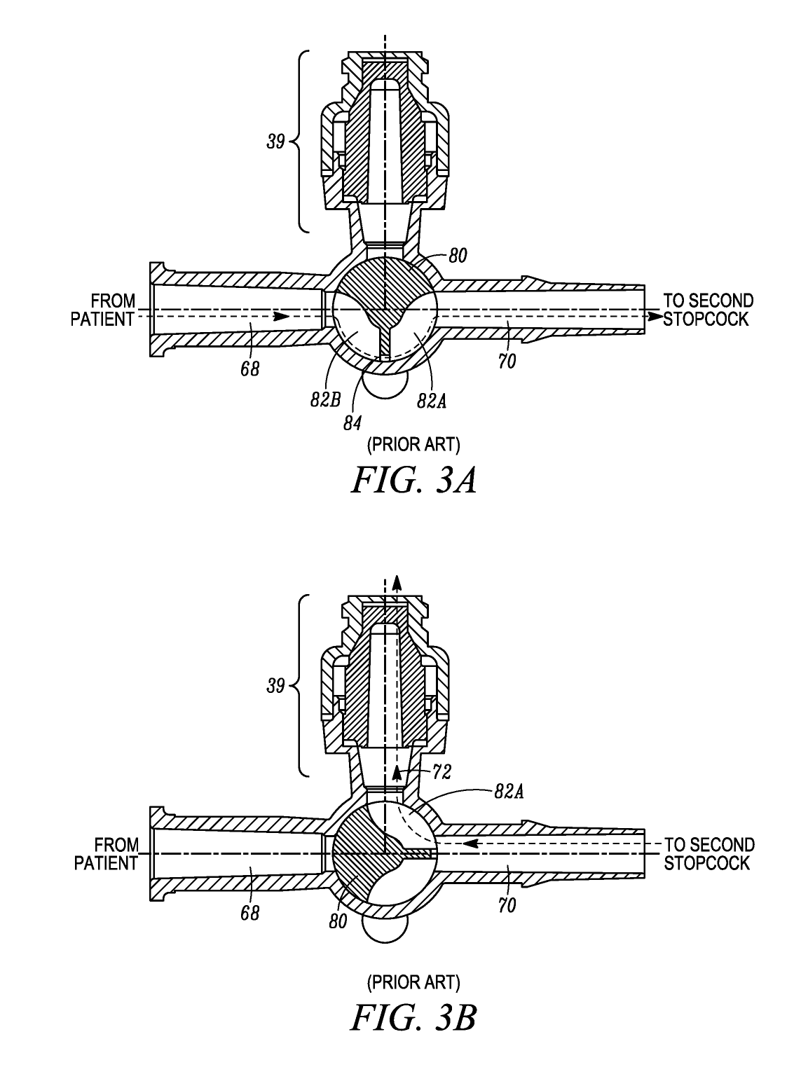

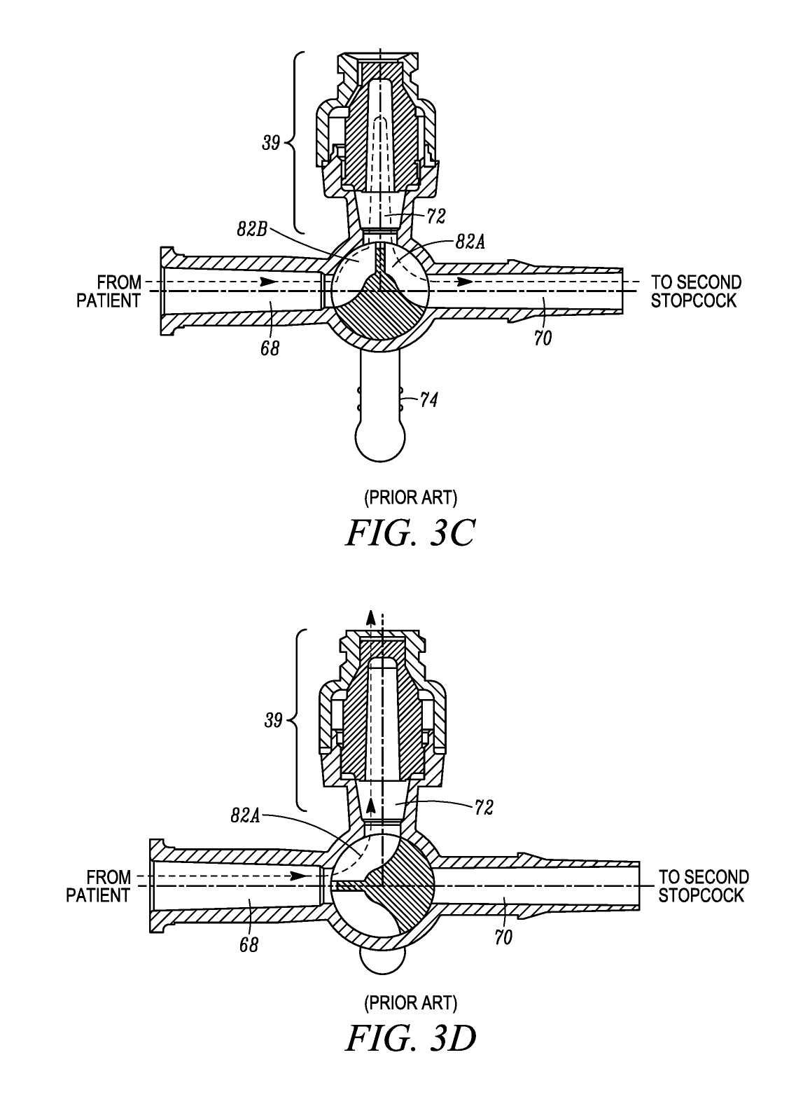

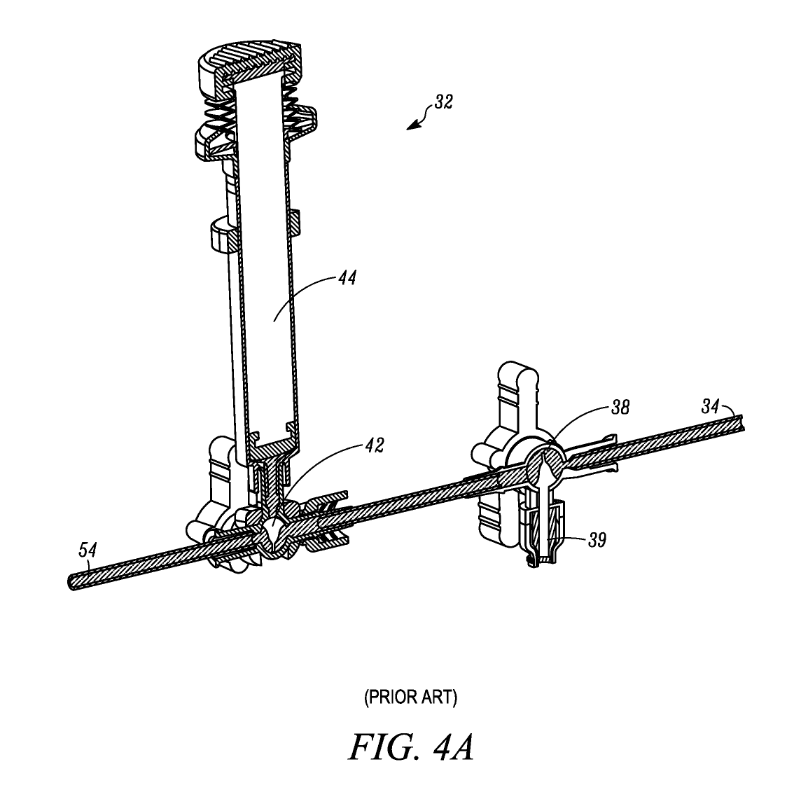

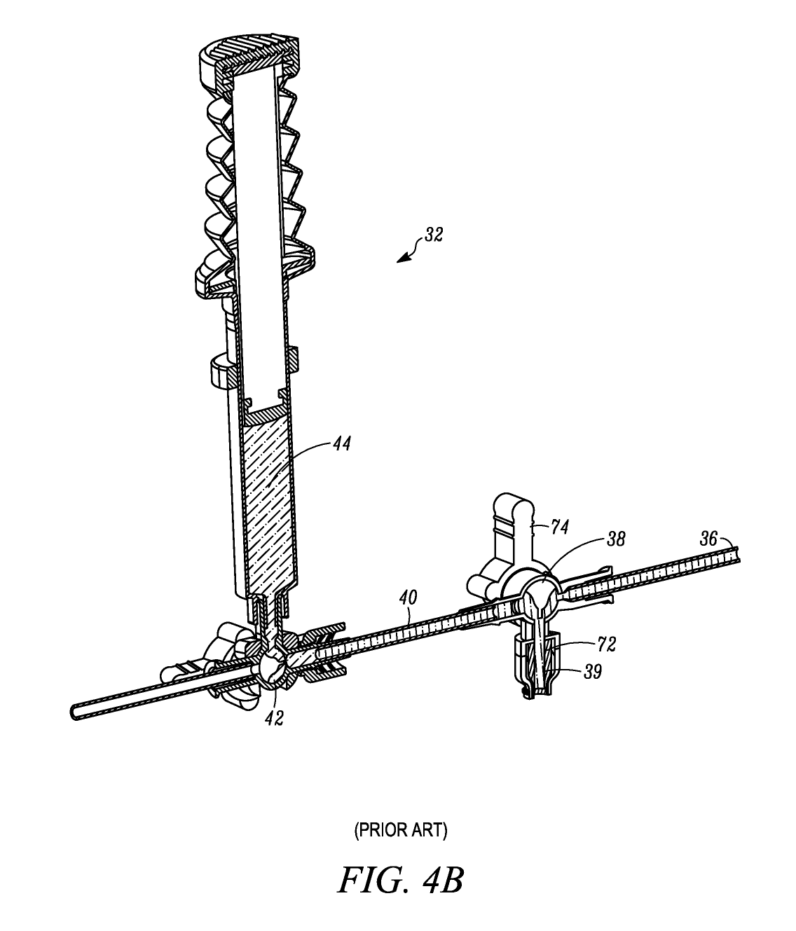

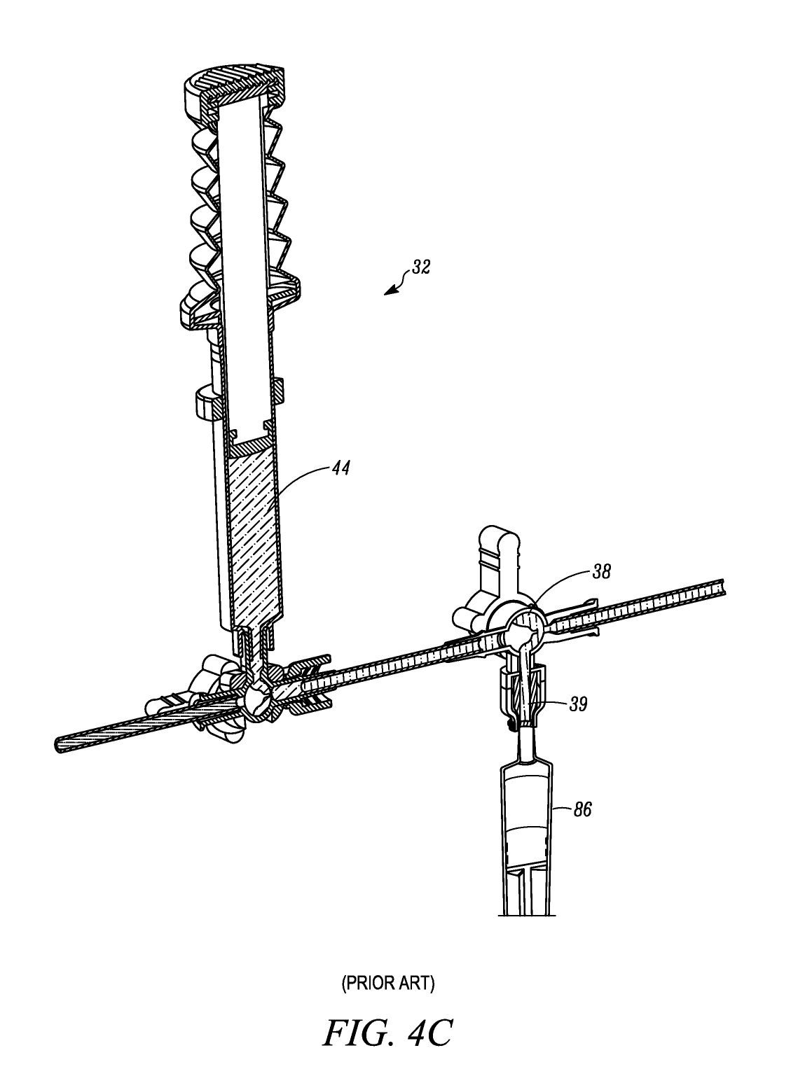

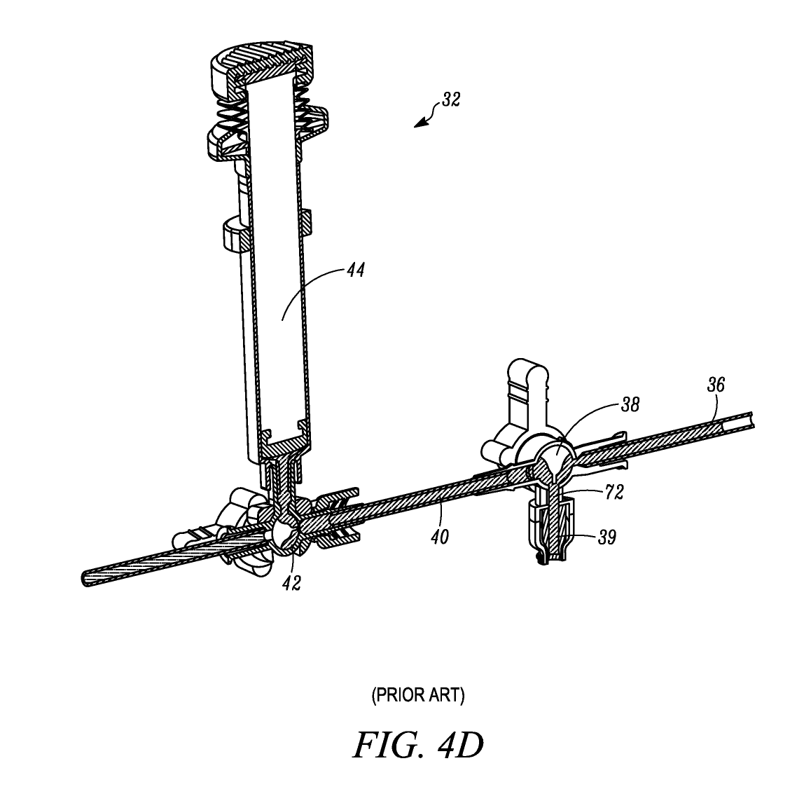

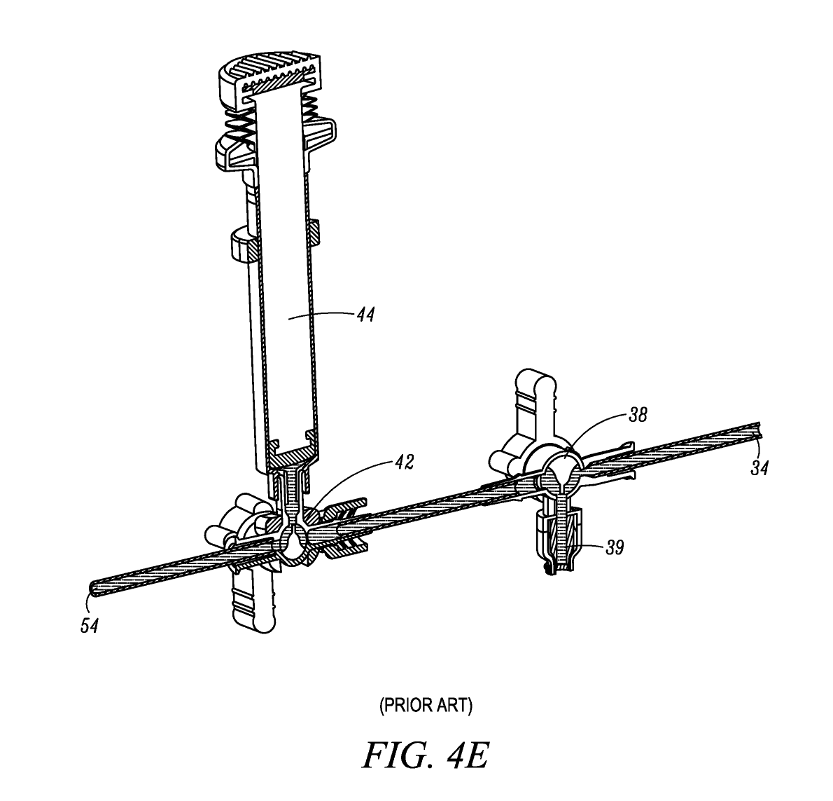

Referring to FIGS. 1 and 4A-E, a conventional blood sampling system 32 is depicted. Referring to FIGS. 2-3D, a conventional stopcock 38 and sampling port assembly 39 is depicted. Details of the conventional blood sampling system 32 and conventional stopcock 38 and sampling port assembly 39 are described in the Background section above.

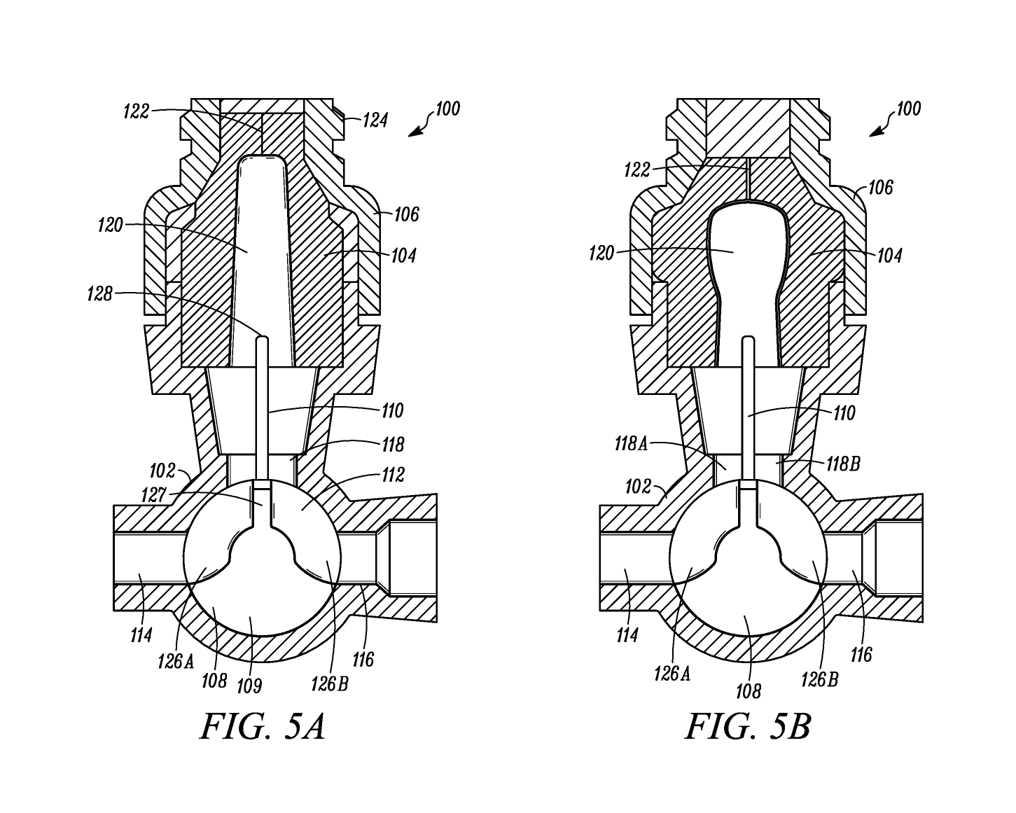

Referring to FIGS. 5A-B, a stopcock and sampling port device 100 is depicted in accordance with an embodiment of the disclosure. The stopcock and sampling port device 100 generally includes a housing 102, and elastomeric element 104, a cap 106, a handle element 108, and a septum 110.

In one embodiment, the housing 102 can define a housing internal fluid passageway 112. Housing internal fluid passageway 112 can be configured as a conduit or channel configured to enable a flow of fluid therethrough. The housing internal fluid passageway 112 can define a first port 114, a second port 116, and a third port 118.

In one embodiment, the housing internal fluid passageway 112 can be substantially cylindrical and can be configured to receive a portion 109 of the handle element 108, such that the handle element 108 can rotate relative to the housing 102. The portion 109 can define a pair of channels or grooves 126a/b, with a divider 127 positioned therebetween. In some embodiments, the divider 127 can be scalloped to enable the passage of fluid between groove 126a and groove 126b when the divider 127 is positioned against a contiguous surface.

The elastomeric element 104 can define an elastomeric element internal fluid passageway 120. The elastomeric element internal fluid passageway 120 can be in fluid communication with the housing internal fluid passageway 112, for example via third port 118. An aperture 122 defined within the elastomeric element 104 can be positioned at one end of the housing internal fluid passageway 112.

The elastomeric element 104 can be constructed of a resilient material, in order to enable the elastomeric element 104 to transition between a relaxed position (as depicted in FIG. 5A) and a compressed position (as depicted in FIG. 5B). The elastomeric element 104 can normally be in a relaxed position. The elastomeric element 104 can be compressed into the compressed position upon the insertion of, for example, a blunt-tipped cannula syringe. The elastomeric element 104 can be configured to bias the aperture 122 closed in the relaxed position, thereby sealing the elastomeric element internal fluid passageway 120 from the outside environment. By contrast, the elastomeric element 104 can be configured to bias the aperture 122 open in the compressed position, thereby enabling a clinician to withdraw fluid within the elastomeric element internal fluid passageway 120 upon compression of the elastomeric element 104.

The cap 106 can operably couple the elastomeric element 104 to the housing 102. For example, in one embodiment, the cap 106 at least partially surrounds the elastomeric element 104 and is fixedly coupled to the housing 102 via ultrasonic welding, adhesive, or the like. In one embodiment, the cap 106 can include a female Luer lock coupling 124.

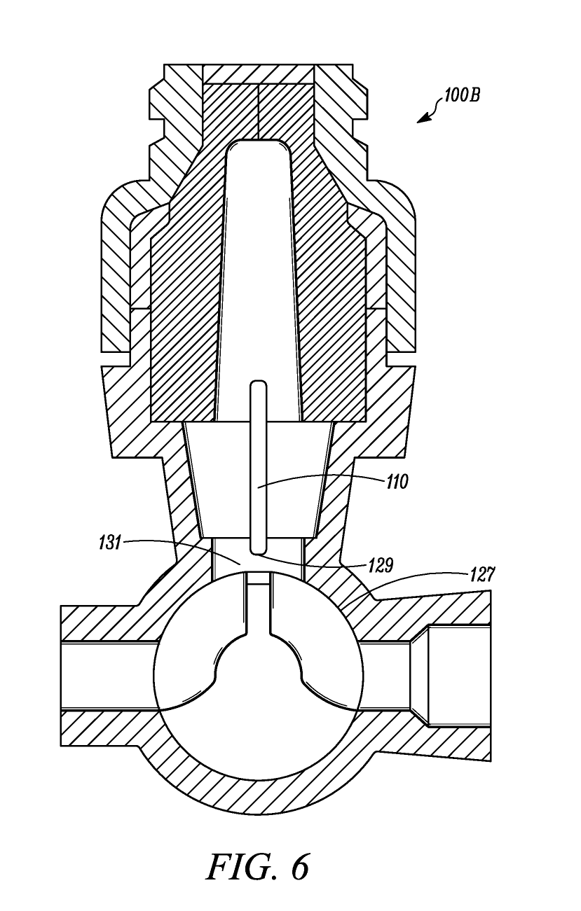

The septum 110 can optionally be operably coupled to the housing 102, within the third port 118, thereby at least partially dividing the third port 118 into a first portion 118a and a second portion 118b. As depicted in FIGS. 5A-B, in one embodiment, the septum 110 can extend into the elastomeric element internal fluid passageway 120, thereby encouraging the flow of fluid within the elastomeric element internal fluid passageway 120 as fluid flowing through the stopcock and sampling port device 100 flows around a terminal end 128 of the septum 110. Accordingly, in one embodiment, the septum 110 extending into the elastomeric element internal fluid passageway 120 reduces the occurrence of stagnation of fluid within the elastomeric element internal fluid passageway 120 by promoting a continuous flow of fluid therethrough during use. In other embodiments, the septum 110 does not extend into the elastomeric element internal fluid passageway 120. As depicted in FIG. 6, in one embodiment, a gap 131 can be defined between an end 129 of the septum 110 and the divider 127, thereby enabling a portion of the fluid flow to pass therethrough. In yet other embodiments, the stopcock and sampling port device 100 does not include a septum 110.

In one embodiment, the handle element 108 is rotatable between a first position, a second position, a third position (as depicted in FIGS. 5A-B), and a fourth position. In the first position, the handle element 108 can be rotated to block or occlude and/or inhibit flow through the third port 118. In the second position, the handle element 108 can be rotated to block or occlude and/or inhibit flow through the second port 116. In the third position, a flow of fluid can be directed through the first port 114, the first channel 126a, the first portion of third port 118a, the elastomeric element internal fluid passageway 120, the second portion of the third port 118b, the second channel 126b, and the second port 116, or vice versa. In the fourth position, the handle element 108 can be rotated to block or occlude and/or inhibit flow through the first port 114.

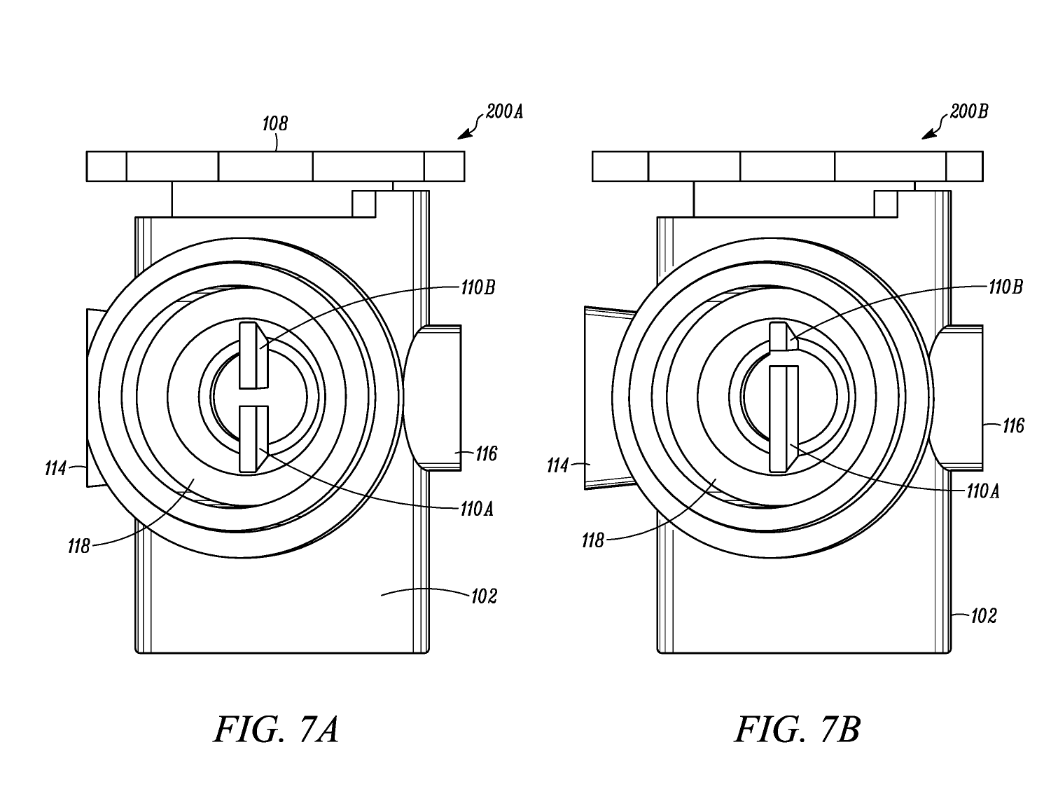

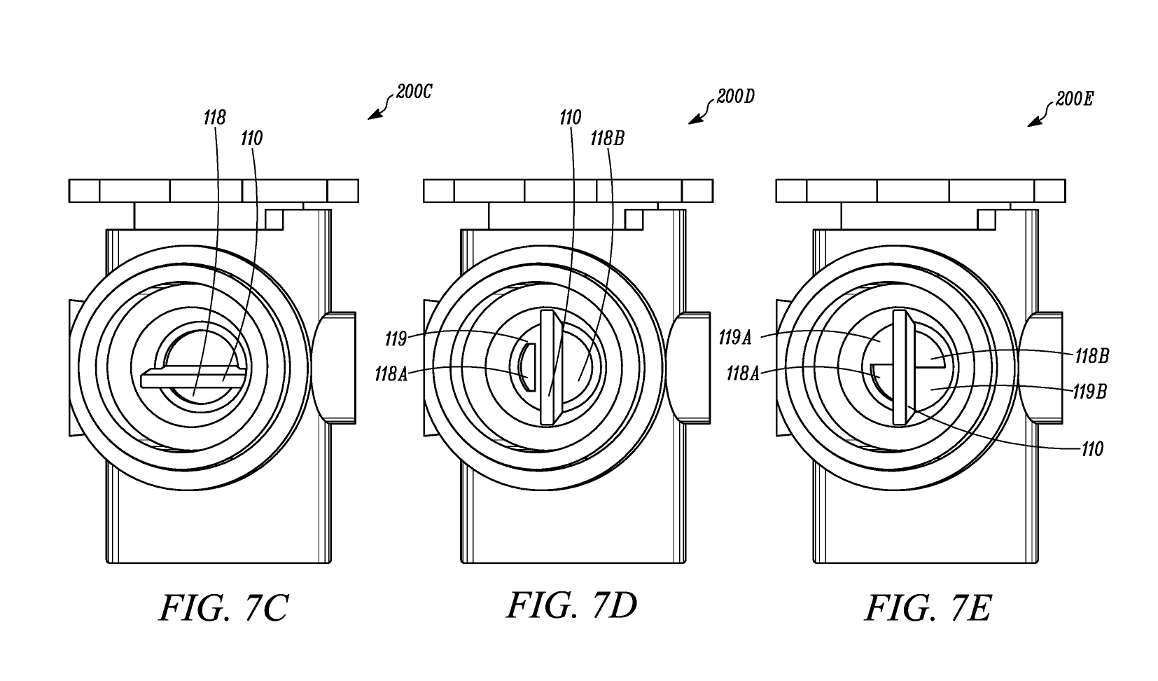

Referring to FIGS. 7A-B, partial, perspective views of stopcock and sampling port devices 200a-b having divided septums 110 are depicted in accordance with embodiments of the disclosure. As depicted in FIGS. 7A-B, the elastomeric element 104 and cap 106 are removed for better viewing of the divided septums 110. The septum 110 can be divided into a first portion 110a and a second portion 110b. Collectively, the two portions 110a/b can be referred to as a divided septum 110. In one embodiment, the two portions 110a/b can be substantially similar in size and shape. In another embodiment, the one portion 110a can be larger than the other portion 110b. In one embodiment, the divided septum 110 can be configured to encourage turbulence of fluid passing through the third port 118 and into the elastomeric element internal fluid passageway 120, thereby reducing the occurrence of stagnation of fluid within the stopcock and sampling port device 100.

Referring to FIGS. 7C-E, additional, partial, perspective views of stopcock and sampling port devices 200c-e are depicted in accordance with embodiments of the disclosure. In these embodiments, other modifications to the septum 110 and/or the first and second portions of the third port 118a/b can be made to encourage turbulence of fluid passing into the elastomeric element internal fluid passageway 120 to reduce the occurrence of stagnation. For example, as depicted in FIG. 7C, in one embodiment, the septum 110 can be rotated approximately 90° relative to the third port 118, so to be substantially orthogonal to the divider 127 of the handle element 108. Other angular offsets between the septum 110 and the divider 127 are also contemplated. As depicted in FIG. 7D, in one embodiment, the first portion of third port 118a can be partially occluded or restricted by a restricting plate 119, so as to encourage an increase in velocity of fluid flowing into the elastomeric element internal fluid passageway 120. As depicted in FIG. 7E, in one embodiment, both the first portion of the third port 118a and the second portion of the third port 118b can be partially occluded or restricted respectively by restricting plates 119a/b, so as to encourage a swirling motion or turbulence of the fluid passing into the elastomeric element internal fluid passageway 120. For example, a top portion of the first portion of the third port 118a can be occluded, and a bottom portion of the second portion of the third port 118b can be occluded, or vice versa. Other configurations to promote turbulence and reduce the occurrence of stagnation of fluid within the stopcock and sampling port device 100 are also contemplated.

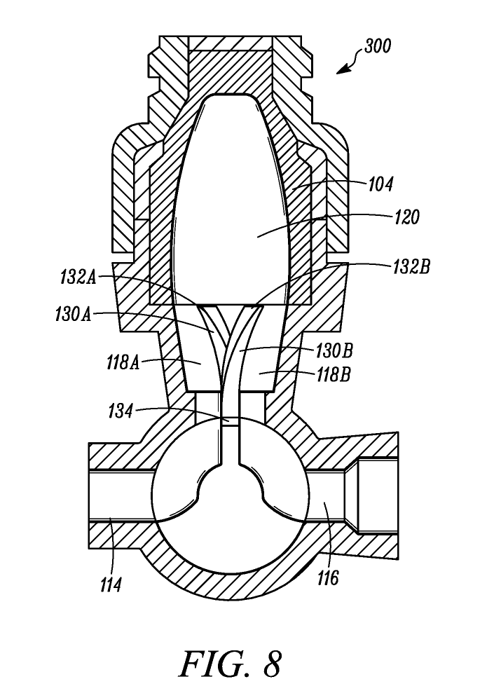

Referring to FIG. 8, a partial cross sectional view of a stopcock and sampling port device 300 having a pair of fluid guide vanes 130a/b is depicted in accordance with an embodiment of the disclosure. In one embodiment, the septum 110 can be replaced by at least one fluid guide vein 130. In one embodiment, the fluid guide vein 130 can be divided into a first portion 130a and a second portion 130b. Like the divided septum 110a/b in the previous embodiments, the first and second portions of the guide vein 130a/b can be substantially similar in size and shape, or one portion can be larger than the other portion. As depicted, the fluid guide veins 130a/b can be shaped for the purpose of guiding a flow of fluid passing therethrough. For example, in one embodiment, the fluid guide veins 130a/b can be curved. In other embodiments, the fluid guide veins 130a/b can be substantially linear or straight, and positioned at an angle with respect to the opening of the third port 118. In one embodiment, a terminal end 132a/b of the fluid guide vein 130 can be in closer proximity to the opening defining the third port 118 than a base 134 of fluid guide vein 130, thereby creating a nozzle configured to affect a change in velocity and/or pressure of fluid flowing through portions of the third port 118. In one embodiment, the first and second portion of the guide vein 103a/b can be configured to impart a swirling motion of fluid passing through the third port 118, thereby reducing the occurrence of stagnation of fluid within the elastomeric element internal fluid passageway 120.

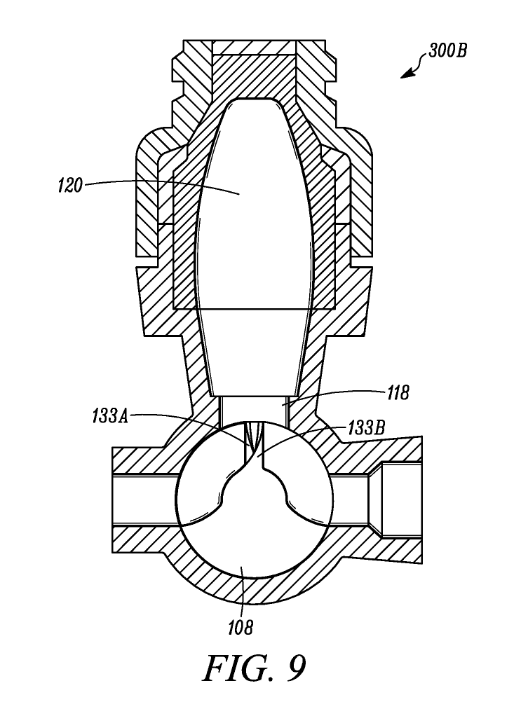

Referring to FIG. 9, a partial cross sectional view of a stopcock and sampling device 300b having a pair of fluid guide veins 133a/b is depicted in accordance with an embodiment of the disclosure. In one embodiment, the divider 127 of handle element 108 can be replaced by at least one fluid guide vein 133. In one embodiment, the fluid guide vein 133 can be divided into a first portion 133a and a second portion 133b. Like the fluid guide vanes 130a/b in previous embodiments, the first and second portions of the guide vein 133a/b can be substantially similar in size and shape, or one portion can be larger than the other portion. As depicted, the fluid guide veins 133a/b can be shaped and sized for the purpose of guiding flow of fluid passing therethrough. For example, in one embodiment, the fluid guide veins 133a/b can be curved. In other embodiments, the fluid guide veins 133a/b can be substantially linear or straight, and positioned at an angle with respect to the third port 118. In one embodiment, the guide veins 133a/b can be configured to impart a swirling motion of fluid passing into the third port 118, thereby reducing the occurrence of stagnation of fluid within the elastomeric element internal fluid passageway.

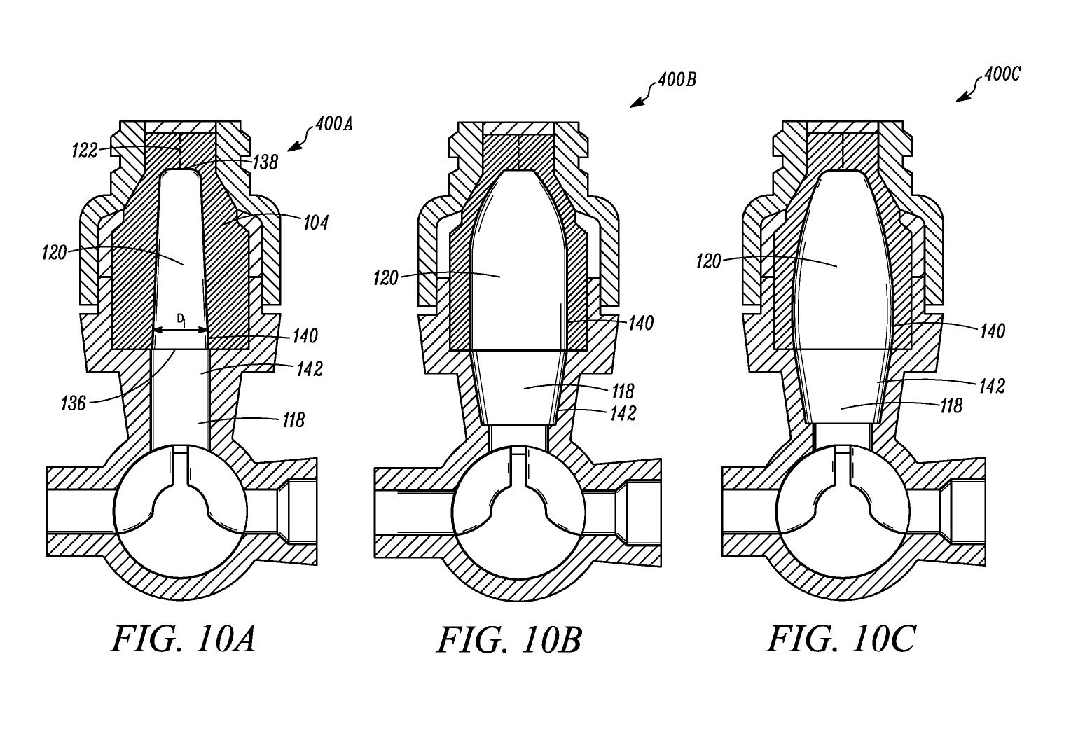

Referring to FIGS. 10A-C, partial cross sectional views of stopcock and sampling port devices 400A-C having smooth transitions between the walls 140 defining the elastomeric element internal fluid passageways 120 and the walls 142 of the third ports 118 are depicted in accordance with embodiments of the disclosure. The elastomeric element internal fluid passageway 120 can have an internal diameter D₁. In one embodiment, the internal diameter D₁ can vary based on its distance between a proximal end 136 and a distal end 138 of the elastomeric element internal fluid passageway 122. For example, as depicted in FIG. 10A, in one embodiment, the internal diameter D₁ can decrease substantially linearly between the proximal end 136 and the distal end 138. In other embodiments, as depicted in FIGS. 10B-C, the internal diameter Dᵢ can increase or decrease along a curved path.

In one embodiment, the internal wall 142 defining the third port 118 can be shaped and sized to create a substantially smooth transition with the internal wall 140 of a standard sized elastomeric element internal fluid passageway 120. In one embodiment, the internal wall 140 defining the elastomeric element internal fluid passageway 120 can be shaped and sized to create a substantially smooth transition with the internal wall 142 defining the third port 118. Accordingly, any step and/or corners between the elastomeric element internal fluid passageway 120 and the third port 118 where stagnation is most likely to occur, can be reduced or eliminated, thereby enabling a smooth flow and a reduction in the occurrence stagnation within the stopcock and sampling port device 100.

In one embodiment, the internal wall 140 and/or internal wall 142 can be shaped to further reduce the occurrence of stagnation. For example, in one embodiment, the internal wall 142 defining the third port 118 can be flush with and/or positioned tangentially to the internal wall 140 of the internal fluid passageway 120, so as to enhance flow. In another embodiment, internal walls 140 and/or 142 can include a plurality of turbulent inducing knobs. In another embodiment, internal walls 140 and/or 142 can include one or more angled ribs and/or threaded textures or patterns to promote a swirling of fluid flowing therethrough.

Examples of catheters include central venous line catheters which, for example, can be placed into the right subclavian vein, or arterial line catheters which can be inserted into an artery. Various example embodiments of catheters are described herein for use in accessing the subclavian veins and arteries of the patient or subject. It is to be appreciated, however, that the example embodiments described herein can alternatively be used to access veins and other blood vessels on a patient. It is additionally to be appreciated that the term “clinician” refers to any individual that can perform the medical and/or blood collection procedure with any of the example embodiments described herein or alternative combinations thereof. Similarly, the term “patient” or “subject” as used herein, is understood to refer to an individual or an object in which the catheter is to be inserted, whether human, animal, or inanimate. Various descriptions are made herein, for the sake of convenience, with respect to the procedures being performed by the clinicians to access the vein of the subject, while the disclosure is not limited in this respect.

Persons of ordinary skill in the relevant arts will recognize that embodiments may comprise fewer features than illustrated in any individual embodiment described above. The embodiments described herein are not meant to be an exhaustive presentation of the ways in which the various features may be combined. Accordingly, the embodiments are not mutually exclusive combinations of features; rather, embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted. Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended. Furthermore, it is intended also to include features of a claim in any other independent claim even if this claim is not directly made dependent to the independent claim.

Moreover, reference in the specification to “one embodiment,” “an embodiment,” or “some embodiments” means that a particular feature, structure, or characteristic, described in connection with the embodiment, is included in at least one embodiment of the teaching. The appearances of the phrase “in one embodiment” in various places in the specification are not necessarily all referring to the same embodiment.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of Section 112, sixth paragraph of 35 U.S.C. are not to be invoked unless the specific terms “means for” or “step for” are recited in a claim.

1. A stopcock and sampling port device configured to reduce the occurrence of stagnation of fluid flowing therethrough, the stopcock and sampling port device comprising:
a housing defining a housing internal fluid passageway having a first port, a second port, and a third port;
an elastomeric element defining an elastomeric element internal fluid passageway and an aperture, the elastomeric element configured to bias the aperture closed in a relaxed position, and bias the aperture open in a compressed position;
a cap configured to operably couple the elastomeric element to the housing; and
a handle rotatably positioned within the housing internal fluid passageway and configured to selectively enable flow between the first port and the second port via the elastomeric element internal fluid passageway;
a septum operably coupled to the housing within the third port and extending into the elastomeric element internal fluid passageway, the septum configured to encourage fluid passing through the third port to flow through the elastomeric element internal fluid passageway, thereby reducing the occurrence of stagnation of fluid within the elastomeric element internal fluid passageway.

2. The stopcock and sampling port device of claim 1, wherein the septum is divided so as to encourage turbulence in fluid passing through the third port.

3. The stopcock and sampling port device of claim 1, wherein at least one of the septum and a divider to handle is shaped and/or angled so as to impart a swirling motion of fluid passing through the third port.

4. The stopcock and sampling port device of claim 1, wherein at least a portion of the third port is partially occluded so as to impart turbulence of fluid passing therethrough.

5. The stopcock and sampling port device of claim 1, wherein the elastomeric element internal fluid passageway and the third port of the housing are shaped and sized to create a smooth transition therebetween.

6. The stopcock and sampling port device of claim 1, wherein the elastomeric element internal fluid passageway has a diameter larger than a diameter of at least one of the first port and the second port.
